(19) United States
(12) Patent Application Publication
(10) Pub. No.: US 2022/0225935 A1
(43) Pub. Date: Jul. 21, 2022

(54) SYSTEM AND METHOD FOR ATHLETE PHYSIOLOGY MONITORING

(71) Applicant: University of Maryland, Baltimore, Baltimore, MD (US)

(72) Inventors: Radi Masri, Columbia, MD (US); James Edward Whitney, II, Baltimore, MD (US); Michael Wright, Baltimore, MD (US)

(21) Appl. No.: 17/716,324

(22) Filed: Apr. 8, 2022

Related U.S. Application Data
(63) Continuation of application No. 16/582,071, filed on Sep. 25, 2019.
(60) Provisional application No. 62/736,015, filed on Sep. 25, 2018.

(51) Int. Cl. A61B 5/00; G16H 40/67; A61B 5/0205; A63B 71/08

(52) U.S. Cl. CPC A61B 5/682; G16H 40/67; A61B 5/02055; A61B 5/6803; A61B 5/02438; A63B 71/085; A61B 5/7282; A61B 5/746; A61B 2503/10; A61B 5/0002

(57) ABSTRACT

Disclosed is a smart, physiology monitoring mouthguard (SPMM) system and method for capturing various physiological data of athletes in real-time for health monitoring. The system is configured to provide real-time and/or recorded physiological data of an athlete to a user, and may be configured to record physiological data of the athlete for later retrieval using sensors and communication systems. Sensors may be chosen based on the anticipated activity or sport, the health and age of the athlete, and size and power requirements of the device. Sensors may include accelerometers, gyroscopes, thermometers, hydrometers, pH, electrolyte (e.g., potassium, sodium, etc.) sensors, and the like. These and other sensors may be configured to measure different physiological data of the athlete, including impact, body temperature, hydration, illness, heart rate, and the like. In some embodiments, sensors on the mouthguard are integrated with external sensors (e.g., accelerometers on a helmet, chest-worn heart rate monitor) on the athlete to provide additional or more accurate physiological data.

### CROSS REFERENCE TO RELATED APPLICATION

This application is a continuation of U.S. application Ser. No. 16/582,071 titled Device for Athlete Physiology Monitoring,” filed Sep. 25, 2019, which claims the benefit of U.S. Provisional Application No. 62/736,015 titled “Device for Athlete Physiology Monitoring,” filed Sep. 25, 2018 by the inventors herein, which application is incorporated herein by reference in its entirety.

### FIELD OF THE INVENTION

The present invention relates generally to systems and methods for monitoring physiological conditions of athletes during athletic events, and more particularly to a system including at least a mouthguard equipped with one or more sensors and processors configured for real-time monitoring of a physiological data of an athlete.

### BACKGROUND

Athletes of all types face risk of injury during practice, training, and competition. For example, professional football players frequently suffer injuries including fractures, sprains, and more dangerous head injuries, such as concussions. Throughout a sporting event, a player may experience a significant impact that jars their head on multiple occasions, creating a cumulative effect that exacerbates a brain injury, possible leading to a concussion or even more serious injury. Similarly, athletes can also be subject to the insidious effects of heat exhaustion and heatstroke. Again, similar to concussions, heatstroke and heat exhaustion are the results on the body due to cumulative exposure to high temperatures and can sometimes go undetected until the athlete is in an emergency situation. However, in the midst of competition, players, coaches, and other team staff may pay limited attention to the building risk of serious injury, leaving the athlete exposed to risk of serious and long-term injury.

It would therefore be advantageous to provide a system and method capable of monitoring the physiological conditions of an athlete in real-time during an athletic event, such that if they experience conditions that present significant risk to their health, team staff may intervene to take them off the field, give them an appropriate rest period, and if necessary seek immediate medical intervention to mitigate the risk of any injury progressing.

### SUMMARY OF THE INVENTION

Disclosed herein is a system and method for capturing various physiological data associated with an athlete in real-time during an athletic event to detect the occurrence of a condition that risks the player's health, which system and method employ an athlete monitoring unit including at least a smart, physiology-monitoring mouthguard (SPMM) device. In certain configurations, the athlete monitoring unit is configured to provide real-time and/or recorded physiological data of an athlete to a user. The athlete monitoring unit may be configured to record physiological data for later retrieval. The athlete monitoring unit may further be configured as a compliant and/or moldable mouthguard, which is generally a convenient form factor for athletes and other users. The athlete monitoring unit may be configured to measure multiple physiologic parameters of an athlete. In certain configurations, each parameter is measured sequentially, while in other configurations, some parameters are measured simultaneously.

In an exemplary embodiment, a system is provided including a mouthguard having a substrate, a computing or data-acquisition platform, sensors, a device-side user interface, a monitoring-side user interface, a communication system, a data storage module, a power module, and software for data and/or signal processing. Some configurations may only include electronic systems on the athlete monitoring unit, such as a substrate, sensors, a power module, and a communication system and/or data storage module. Other configurations may only include electronic systems configured to operate with a mouthguard, such as by attaching sensors, a power module, and a communication system and/or data storage module to a typical mouthguard. Furthermore, electronic systems can include a printed circuit that is directly formed on or into the mouthguard substrate. Thus, the athlete monitoring unit and system can be configured to accommodate many cost and functional requirements.

In accordance with certain aspects of an exemplary embodiment, a system for monitoring the physiological condition of an athlete is provided, comprising: at least one athlete monitoring unit comprising a mouthguard, at least one sensor attached to the mouthguard, wherein the sensor is configured to sense a physiological condition of an athlete on which the athlete monitoring unit is worn, and a microcontroller in data communication with the at least one sensor; and a base station having a processor and a data memory, wherein the base station is in data communication with the athlete monitoring unit, the base station having computer executable code stored thereon configured to receive physiological data from the athlete monitoring unit, compare the physiological data received from the athlete monitoring unit with a preset threshold value for the physiological data, and upon a determination at the base station that the physiological data received from the athlete monitoring unit exceeds the threshold value, generate at the base station a human-discernable alert.

In accordance with further aspects of an exemplary embodiment, a method for monitoring athlete physiological data is provided, comprising: providing at least one athlete monitoring unit comprising a mouthguard, at least one sensor attached to the mouthguard, wherein the sensor is configured to sense a physiological condition of an athlete on which the athlete monitoring unit is worn, and a microcontroller in data communication with the at least one sensor; and providing a base station having a processor and a data memory, wherein the base station is in data communication with the athlete monitoring unit; receiving at the base station physiological data from the athlete monitoring unit; comparing at the base station the physiological data received from the athlete monitoring unit with a preset threshold value for the physiological data; and upon a determination at the base station that the physiological data received from the athlete monitoring unit exceeds the threshold value, generating at the base station a human-discernable alert.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1 is a schematic view of a system for monitoring the physiological condition of an athlete according to certain aspects of an embodiment of the invention.

FIG. 2 is a schematic view of an exemplary user interface for use with the system of FIG. 1.

### DETAILED DESCRIPTION OF THE PREFERRED EMBODIMENTS

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced items.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

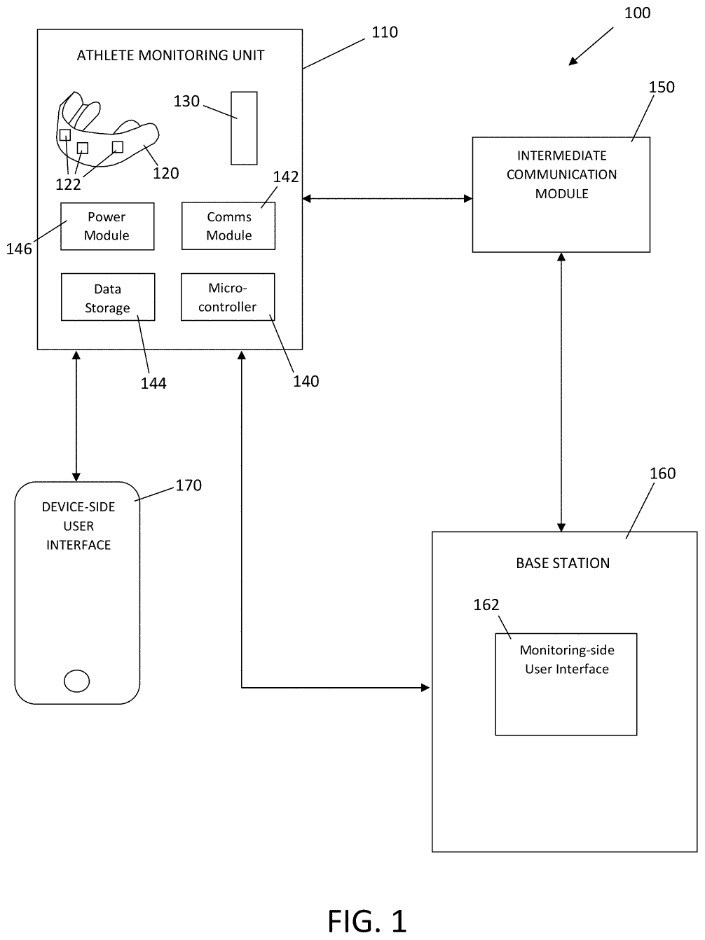

Provided herein is a system and method for capturing various physiological data associated with an athlete in real-time during an athletic event to detect the occurrence of a condition that risks the player's health, which system and method employ a smart, physiology-monitoring mouthguard (SPMM) device. With particular reference to FIG. 1 and in accordance with certain aspects of an exemplary embodiment, system 100 includes at least one athlete monitoring unit 110 including a mouthguard 120 that conforms to an athlete's mouth, and more particularly their upper teeth. Mouthguard 120 includes at least one mouthguard-mounted sensor 122, and optionally a plurality of mouthguard-mounted sensors 122, for detecting physiological conditions experienced by the athlete during an athletic event. In accordance with certain aspects of an embodiment, athlete monitoring unit 110 may include multiple integrated sensors, including both mouthguard-mounted sensors 122 and other body-worn sensors 130, as discussed in greater detail below, that may be customized or selected for each athlete. In certain configurations, athlete monitoring unit 110 includes a microcontroller 140 that is in data communication with sensors 122 and 130. While FIG. 1 shows microcontroller 140 positioned remotely from mouthguard 120, microcontroller 140 may in certain configurations be integrated within mouthguard 120. Microcontroller 140 may receive signals (e.g., signals indicative of physiological conditions experienced by the athlete) from sensors 122 and 130, and may control each such sensor's operation (e.g., turn each sensor on and/or off).

In an exemplary embodiment, the sensors 122 and 130 are multiplexed to microcontroller 140 using, by way of non-limiting example, BLUETOOTH® connectivity, although other wireless or wired communication protocols may likewise be employed as will be apparent to those of ordinary skill in the art. Microcontroller 140 is likewise preferably in data communication with a base station 160 through a communication module 142 of athlete monitoring unit 110. Microcontroller 140 is preferably configured to process physiological data and signals, and to transmit such data and signals to base station 160. Microcontroller 140 is preferably configured to store data in a data storage module 144, transmit such data to base station 160, and/or delete physiological data from data storage module 144, as described in further detail below. Likewise, base station 160 is preferably configured to employ command and control functions (e.g., system 100 set-up or initialization), perform real-time data collection and routing, perform real-time physiology monitoring and alerting, provide a graphical user interface, and perform any desired post-processing of data received from athlete monitoring unit 110.

Preferably, athlete monitoring unit 110 also includes a power module 146, such as a rechargeable battery, for powering the components of athlete monitoring unit 110.

In certain configurations, microcontroller 140 directly communicates with base station 160, while in other configurations microcontroller 140 indirectly communicates with base station 160. Such communications may be wireless or wired communications. For example, microcontroller 140 may wirelessly communicate with base station 160 using a BLUETOOTH® connection (or radio, Wi-Fi, or other wireless connection) employed by communication module 142 in accordance with well-known wireless communication protocols and infrastructure, to transmit physiological data from athlete monitoring unit 110 to base station 160. As a further example, microcontroller 140 may communicate with an intermediate communication module 150 that further communicates with base station 160. Intermediate communication module 150 may, by way of non-limiting example, be a body- or equipment-worn repeater module having a higher power transmitter module and more power capacity (e.g., an additional battery) that can communicate physiological data to base station 160 under a variety of challenging communication environments (e.g., poor signal quality due to noise, relative locations of microcontrollers 140, intermediate modules 150 and/or base stations 160, interference, weather, etc.). Alternatively or additionally, intermediate communication modules 150 may be positioned on an athletic field or other area surrounding the athletes at optimal positions between the athletes and base station 160. Thus, microcontroller 140 may comprise a low-power microcontroller configured to increase the efficiency and reduce the power requirements of athlete monitoring unit 110.

In certain configurations, more than one intermediate communication module 150 may be provided that communicate between athlete monitoring unit 110 and base station 160. In other configurations, each intermediate communication module 150 and/or microcontroller 140 may only communicate with predetermined intermediate communication modules and/or base stations 150. In still other configurations, each intermediate communication module 150 and/or microcontroller 140 may communicate with a variable set of intermediate communication modules 150 and/or base stations 160 depending upon a variety of factors (e.g., signal quality due to noise, relative locations of microcontrollers 140, intermediate communication modules 150, and/or base stations 160, interference, weather, etc.). By way of non-limiting example, each microcontroller 140 and/or intermediate communication module 150 may communicate with intermediate communication modules 150 that are spatially close to the athlete's athlete monitoring unit 110 at particular moments.

In other configurations, system 100 many include multiple base stations 160. In some configurations, base station 160 may comprise a smartphone or tablet device, while in other configurations base station 160 may comprise a desktop computer, laptop, receiver, or the like. Base station 160 may be configured to record and display incoming data. In another configuration, base station 160 may be configured to activate alerts when triggered by particular physiological data or conditions. For example, for a device measuring physiological data including high acceleration (high-G-force) or impacts to an athlete (e.g., from an athlete monitoring unit 110 having a sensor 122 and/or 130 for measuring acceleration such as an accelerometer or gyroscope, as further described below), a base station 160 may be configured to issue an alert when the athlete experiences a certain number of occurrences within a predetermined duration (e.g., a single sporting event). The alert may indicate a change of the athlete's status, for example, from “unmarked” to “in-danger,” such that coaches, health professionals, authorities, etc. can intervene as necessary. Other configurations of the system can include similar algorithms for other sensors (as further described below).

In one configuration, the system 100 monitors for concussions of an athlete. For example, the system 100 monitors for concussions by measuring impact forces via acceleration measurements. Athlete monitoring unit 110 may include a three-axis accelerometer to measure acceleration, which can be recorded directly in data storage module 144, which may comprise (by way of non-limiting example) a non-volatile memory. In other configurations, data can be streamed or transmitted to remote base station 160, which may comprise (by way of non-limiting example) an off-field central team data collection system (as described above; see also Table 2, below). In still other configurations, the acceleration data can be simultaneously streamed or transmitted to base station 160 and recorded in a storage module at base station 160.

In an exemplary embodiment, acceleration data is only recorded or streamed when the accelerometer(s) of athlete monitoring unit 110 measure an impact force exceeding a pre-set threshold (on-demand operation), such as an impact event. For example, data may only be recorded or streamed for a collection interval after an impact event. The collection interval for each impact event can last from several seconds preceding the event and throughout the event, to a time after the event. For example, the collection interval may be 1 second before the event and 5 seconds after the event, and in another example, the collection interval may be 2 seconds before the impact event and 10 seconds after the impact event. The system may readily be adapted to provide for longer or shorter data collection durations as may be deemed appropriate for a particular athletic event. Each occurrence of an impact event may be time-stamped, processed, or synced at one of the base stations 160. Thus, operating on-demand may reduce power consumption of the device and increase battery life of the device.

In an exemplary embodiment, the substrate of mouthguard 120 is similar to a typical mouthguard, such as those used during athletic activities (e.g., football). Some mouthguards include an attachment strap that is configured to couple to a helmet or jersey. Thus, some configurations herein may include wiring that is integrated with an attachment strap attaching mouthguard 120 to the athlete's helmet, jersey, pads, or other equipment for power module and/or communication systems further integrated with the athlete's helmet, jersey, pads, or other equipment (such as described below). In another configuration, the mouthguard may be custom designed for a particular athlete, and in yet another configuration the mouthguard may be custom designed to house electronic systems of the device-side platform (such as the microcontroller, sensors, etc.) to protect the electronic systems while allowing sufficient measurement of physiological data and safety of the athlete. The mouthguard 120 may be manufactured according to typical methods, such as injection molding, additive manufacturing, and the like.

In accordance with certain features of an exemplary embodiment, sensors 122 and 130 may be configured to measure physiological data of the athlete. The sensors 122 and 130 are configured to sufficiently and reliably provide signals for monitoring the health of the athlete. Different types of sensors 122 and 130 may be chosen based on the anticipated activity or sport, the health and age of the athlete, and size and power requirements of the system 100. For example, the sensors 122 and 130 may include accelerometers (e.g., multiple three-axis accelerometers, as discussed above), gyroscopes, thermometers, hydrometers, pH sensors, electrolyte (e.g., potassium, sodium, etc.) sensors, heart rate monitors, and the like, the construction of which are known to those skilled in the art. These and other sensors may be configured to measure different physiological data of the athlete, including impact, body temperature, hydration, illness, heart rate, and the like. In some configurations, sensors 122 on the mouthguard 120 are integrated with external sensors 130 on the athlete to provide additional or more accurate physiological data. For example, physiological data from accelerometers on the mouthguard 120 can be integrated with physiological data from accelerometers on the athlete's helmet (e.g., via the microcontroller 140) to more accurately determine impact forces relative to the head. As a further example, physiological data from the mouthguard 120 can be integrated with physiological data from heart rate monitors or temperature sensors on the athlete (e.g., on the athlete's body, jersey, pads, helmet, or the like, via the microcontroller 140) to more accurately determine the athlete's temperature or overall health. Some sensors 122 and 130 may be monitored continuously, while other sensors 122 and 130 may be monitored discretely or intermittently.

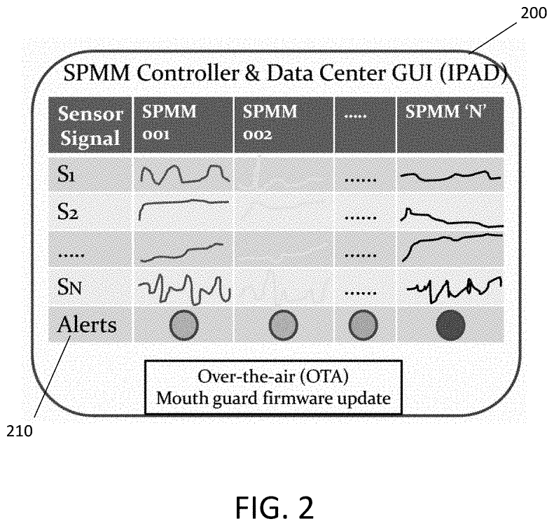

In an exemplary embodiment, system 100 includes at least one base station 160, as discussed above. Base station 160 (or, computing/data-acquisition platform or monitoring-side platform) is preferably configured to receive all of the physiological data transmitted from the athlete monitoring unit 110. Base station 160 may be further configured to process the received physiological data, such as according to a set of pre-defined rules or algorithms. Furthermore, base station 160 may be configured to issue alerts, such as determined by the rules or algorithms regarding the status of the athlete (e.g., “unmarked,” “in danger”, or the like). Still further, some configurations of base station 160 may display the physiological data, along with any alerts, or cause other devices (e.g., smartphone of a healthcare provider) to display alerts or physiological data. FIG. 2 is an exemplary view of a display screen from a user interface of base station 160, which may (by way of non-limiting example) provide for each sensor 122 and 130 of each athlete monitoring unit 110 a display of data (e.g., numeric values, graphs, or other indicia as may be appropriate for the sensed physiological condition) associated with each sensor, along with an “Alerts” indicator 210 associated with each athlete monitoring unit 100 to provide a quickly identifiable indicia of an overall physiological condition of the athlete (e.g., distinct color indicators to indicate unmarked, in danger, and the like). By way of non-limiting example, base station 160 may comprise a smartphone (e.g., an Android®- or iOS®-based phone or tablet), a personal PC or laptop, or the like. Still further, each base station 160 may be configured to receive physiological data from and operate many athlete monitoring units 110 at a particular time.

In an exemplary embodiment, system 100 may have adjustable settings of the electronics on the athlete monitoring unit 110. For example, the system may include a device-side user interface (UI) 170, such as a graphical user interface, in data communication with athlete monitoring unit 110. The device-side UI 170 may be configured to adjust a selection of many settings, such as change data signal sensitivity, trigger thresholds, device initialization (e.g., how to turn on each athlete monitoring unit 110 or start data monitoring/acquisition), setup parameters, establishing a unique identification (an identifiable tag, e.g., a radiofrequency or bar code tag) for each athlete monitoring unit per athlete, and the like. In an exemplary embodiment, the device-side UI 170 operates on a PC or laptop (although other embodiments operate on a smartphone, tablet, or the like), which transmits settings to the athlete monitoring unit 110. While FIG. 1 shows device side UI 170 implemented on a computing device that is separate from base station 160, device side UI 170 may alternatively be operated as an executable software module on base station 160 without departing from the spirit and scope of the invention.

In an exemplary embodiment, the system 100 may also have adjustable settings of the base station 160 (monitoring-side). For example, the system 100 may include a monitoring side user interface 162, such as a graphical user interface, on the base station 160 (as depicted in FIG. 2). The monitoring-side UI 162 may be configured to receive, process, and transmit physiological data, initialize other components (e.g., individual athlete monitoring units 110, intermediate modules 150, other base stations 160, etc.), start and/or stop data monitoring or acquisition, raise data-dependent alerts, maintain time-base for athlete monitoring units 110, monitor battery levels, initiate communications with healthcare providers, register individual athlete monitoring units 110 (e.g., using RFID), and the like.

In an exemplary embodiment, the communications system comprises a BLUETOOTH® low-energy (BLE) system. For example, typical BLE systems have approximate transmission capabilities shown in Table 1, below. In some configurations that require long transmission distances, such as for motorsport applications, higher-powered alternative technologies (e.g., radio transmission) can be used. In other configurations, the communications system can operate with external repeaters in communication with higher-powered radios or other BLE devices. In still other configurations, a wireless sensor network (WSN) architecture can be used for connectivity to the base station 160.

TABLE 1
Bluetooth Class, Power, and Range

| Device Class | Transmit Power | | Intended Range |
|---|---|---|---|
| Class 3 | 1 | mW | less than 10 meters |
| Class 2 | 2.5 | mW | 10 meters, (33 feet) |
| Class 1 | 100 | mW | 100 meters (330 Ft.) |

In an exemplary embodiment, as discussed above, the data storage module 144 is configured to store physiological data on the athlete monitoring unit 110. The data storage module 144 may comprise different types of storage, such as on-board random-access-memory, microSD cards (e.g., permanently installed), and may be supplemented by storage at the base station 160, and/or storage on a remote server (e.g., the cloud). Alternatively, all data may be stored at the base station 160 and/or storage on a remote server instead of being worn by that athlete as a part of athlete monitoring unit 110. Physiological data may be stored (or synced to a base station 160) in the data storage modules 144 continuously or intermittently depending on many factors, such as power capacity, range from a base station 160, physiological data and respective thresholds, etc. For example, the athlete monitoring unit 110 may store (record) and communicate physiological data for a duration before, during, and after an impact event (as described above). Furthermore, the athlete monitoring unit 110 may be configured to store or communicate data periodically. Still further, the athlete monitoring unit 110 may be configured to store (record) or communicate data according to other schemes that would be understood by persons of ordinary skill in the art.

In an exemplary embodiment, the power module 146 of the athlete monitoring unit 110 is configured to have sufficient power to support operations of the athlete monitoring unit 110 for at least one full sporting event (e.g., a football game). The amount of power consumption is generally a function of the operations of the athlete monitoring unit 110, such as sampling rates of the sensors 122 and 130 and the communications requirements (e.g., active transmission rate, “on” time, distance from base station 160, obstructions and interference, etc.). For example, during athletic events with venues (e.g., soccer, football, lacrosse, and the like), direct communication from the athlete monitoring unit 110 to the base station 160 can be enabled via BLE. Table 2, below, describes typical dimensions of some exemplary athletic activities. Thus, the power module 146 of athlete monitoring unit 110 may include an on-board battery, such as a button battery or a lithium-ion battery. As a further example, for athletic events with a larger venue or field-of-play (e.g., motorsports) that require additional power, the power module 146 of athlete monitoring unit 110 may include an on-board battery and an auxiliary battery (e.g., at least one lithium-ion battery) on the athlete (e.g., in the helmet, jersey, pads, or the like) that is coupled to an intermediate communication module 150, such as a repeater, for long distance communication to a base station 160 (such as described above). By way of non-limiting example, such a repeater may be a Class 1 BLUETOOTH® module, or other mid-range communications module, as described above. In at least some configurations, each of the batteries are rechargeable. In some configurations, the batteries are rechargeable using an induction charging system. In still other configurations, the athlete monitoring unit 110 is configured to scavenge or harvest energy, such as from the athlete, the athlete monitoring unit 110, and/or the environment, to recharge batteries and/or power the device. For example, the athlete monitoring unit 110 may include piezoelectric materials that generate electric voltage in response to mechanical stresses, such as by an athlete biting the mouthguard 120.

TABLE 2
Dimensions of Various Sports Venues₁

| Dimensions Venue | Length, Ft. | Width, Ft. |
|---|---|---|
| Football field | 360 | 160 |
| Soccer Field | 330 ≤ L ≤ 363 | 211 ≤ W ≤ 248 |
| Basketball Court | 94 | 50 |
| (NBA) | | |
| Tennis Court₂ | 78 | 27 |
| Lacrosse | 330 | 180 |
| Baseball (Outfield | 250 ≤ OW ≤ 421 | NA |
| Wall) | | |
| Hockey | 200 | 85 |
| Track & Field₃ | 216 | 108 |

NOTES:
₁It should be noted that for sports where the base station can be located on the field-of-play center-line, the required transmission distance is only +/− half of the court dimension, i.e., for a football application with the base station located on the center line the required transmission range is approximately +/− 180 feet. In this case, the 180 ft. dimension fits readily within the 330 ft. BLE specification, whereas the 360 ft. field dimension does not.
₂Tennis court widths for ‘doubles’ matches extends to 36 ft.
₃Track median dimensions.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. Thus, it should be understood, therefore, that the invention may be practiced otherwise than as specifically set forth herein.

What is claimed is:

1. A system for monitoring the physiological condition of an athlete, comprising:
at least one athlete monitoring unit comprising:
a mouthguard;
at least one sensor attached to said mouthguard, wherein said sensor is configured to sense a physiological condition of an athlete on which said athlete monitoring unit is worn;
a data storage module;
a communication module; and
a microcontroller in data communication with said at least one sensor, said microcontroller further comprising a processor, wherein said processor of said microcontroller further comprises computer executable code stored thereon configured to:
determine at said microcontroller whether data received from said at least one sensor at a specific time exceeds a threshold value; and
in response to a determination that said data received from said at least one sensor at a specific time exceeds said threshold value, change an operating mode of said data storage module from a non-recording mode to a recording mode to record at said data storage module data received from said at least one sensor, and cause said communication module to transmit to a base station a collection of said data received from said at least one sensor and stored in said data storage module, wherein said collection of said data further comprises data from during and after said specific time; and
said base station having a processor and a data memory, wherein said base station is in data communication with said athlete monitoring unit, said base station having computer executable code stored thereon configured to:
receive physiological data from said athlete monitoring unit;
compare said physiological data received from said athlete monitoring unit with a preset threshold value for said physiological data; and
upon a determination at said base station that said physiological data received from said athlete monitoring unit exceeds said threshold value, generate at said base station a human-discernable alert.

2. The system of claim 1, wherein said computer executable code stored on said base station is further configured to:
determine at said base station whether said physiological data received from said athlete monitoring unit over a specified period of time exceeds a threshold cumulative value for said physiological data; and
upon a determination that said physiological data received form said athlete monitoring unit over a specified period of time exceeds a threshold cumulative value for said physiological data, generate at said base station a human-discernable alert.

3. The system of claim 1, wherein said microcontroller is further configured to delete physiological data from said data storage module during time periods in which said data received from said at least one sensor does not exceed said threshold value.

4. The system of claim 1, wherein said athlete monitoring unit further comprises at least a second sensor attached to equipment worn by said athlete other than said mouthguard.

5. The system of claim 4, wherein said first and second sensors comprise accelerometers.

6. The system of claim 5, wherein said second accelerometer is affixed to a helmet worn by said athlete.

7. The system of claim 4, wherein said first and second sensors are selected from the group consisting of accelerometers, gyroscopes, thermometers, hydrometers, pH sensors, electrolyte sensors, and heart rate monitors.

8. The system of claim 7, wherein said computer executable code on said base station is further configured to detect from data received from said first and second sensors at least one physiological condition of said athlete selected from the group consisting of head impact, body temperature, body hydration, illness, and heart rate.

9. The system of claim 1, further comprising an intermediate communication module in data communication with said athlete monitoring unit and said base station, wherein said intermediate communication module is configured to forward data received from said athlete monitoring unit to said base station.

10. The system of claim 1, wherein said microcontroller is worn by said athlete separate from said mouthguard, said athlete monitoring unit further comprising wiring interconnecting said mouthguard with said microcontroller, wherein said wiring is affixed to an attachment strap extending between said mouthguard and equipment worn by said athlete.

11. The system of claim 1, further comprising a device-side user interface in data communication with said athlete monitoring unit, said device-side user interface having computer executable code configured to:
modify data signal sensitivity at said athlete monitoring unit;
set threshold data levels at said athlete monitoring unit;
initialize said athlete monitoring unit; and
establish a unique identification for each athlete monitoring unit.

12. The system of claim 11, wherein said computer executable code of said base station is further configured to:
provide at said base station further a monitoring-side user interface, wherein said monitoring-side user interface is further configured to:
start and stop monitoring and data acquisition of said system;
generate data-dependent alerts at said base unit;
maintain a time-base for said athlete monitoring units;
monitor battery levels of one or more batteries in said athlete monitoring units;
initiate communications with healthcare providers in response to data received from said at least one athlete monitoring unit; and
register individual athlete monitoring units to said system.

13. A method for monitoring athlete physiological data, comprising:
providing at least one athlete monitoring unit comprising:
a mouthguard;
at least one sensor attached to said mouthguard, wherein said sensor is configured to sense a physiological condition of an athlete on which said athlete monitoring unit is worn;
a data storage module;
a communication module; and
a microcontroller in data communication with said at least one sensor, said microcontroller further comprising a processor; and
providing a base station having a processor and a data memory, wherein said base station is in data communication with said athlete monitoring unit;
determining at said microcontroller whether data received from said at least one sensor at a specific time exceeds a threshold value;
in response to determining that said data received from said at least one sensor at a specific time exceeds said threshold value, change an operating mode of said data storage module from a non-recording mode to a recording mode to record at said data storage module data received from said at least one sensor, and causing said communication module to transmit to said base station a collection of said data received from said at least one sensor and stored in said data storage module, wherein said collection of said data further comprises data from during and after said specific time;
receiving at said base station physiological data from said athlete monitoring unit;
comparing at said base station said physiological data received from said athlete monitoring unit with a preset threshold value for said physiological data; and
upon a determination at said base station that said physiological data received from said athlete monitoring unit exceeds said threshold value, generating at said base station a human-discernable alert.
